(19) United States
(12) Patent Application Publication
(10) Pub. No.: US 2007/0068806 A1
(43) Pub. Date: Mar. 29, 2007

(54) Biosensor strip having an identification function and its sensor

(75) Inventors: Paul Yang, Chung Ho City (TW); Ya-Hsin Chuang, Taipei City (TW); Shih-Chieh Hsiao, Taipei City (TW); Hsin-Yi Lin, Taitung City (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD, Chung Ho City (TW)

(21) Appl. No.: 11/235,261

(22) Filed: Sep. 27, 2005

(51) Int. Cl. G01N 33/487

(52) U.S. Cl. 204/403.01

(57) ABSTRACT

A biosensor strip having an identification function includes a first insulating substrate and a second insulating substrate, and the first substrate is deposited on the second substrate, and the first substrate has an electrode and an identifying electrode area, and the electrode and identifying electrode area are exposed from the second substrate. The identifying electrode area is made by coating an electrically conductive material. Since different conductive materials have different impedances, the conductive material can be changed according to different testing applications or different sensing instruments. When the invention is used and the strip is inserted into a sensor, the impedance produced by the conductive material of the identifying electrode area or the impedance produced between the electrode and the identifying electrode area is used to determine whether or not the impedance of the sensor falls within the predetermined range; if no, then an error message will be produced to avoid any distortion of the test; if yes, then the biological test sample will be tested. As a result, it is not necessary to change the sensor, and manufacturers only need to change the factory default settings of the impedance. Unlike the prior art strip that requires changing the position and form of the identifying electrode to fit different applications or sensors of different specifications, the invention can save manufacturing cost and provide convenient uses.

### BACKGROUND OF THE INVENTION

1. Field of the Invention

The present invention relates to a biosensor strip having an identification function and its sensor that use the characteristic of different conducting materials having different impedances to coat different conducting materials onto an identifying electrode area of a strip as needed, and it is not necessary to change the specification of the identifying device on the sensor when the position of the identifying area of the strip is changed, and thus it can save costs. When the sensor is used for testing, the set impedance of the sensor is compared with the impedance of the identifying electrode area of the strip. The sensor strip unmatched to the sensor cannot be used for the test, so that an examiner will not misuse the sensor strip or misjudge the test.

2. Description of the Related Art

Since the concept of household nursing becomes increasingly popular, and there are various different kinds of self testing products for a fast, cheap, non-professional operation, and these sensor products include a blood sugar meter, an electronic ear thermometer, and an electronic sphygmomanometer, and the disposable blood sugar strip used for a blood sugar meter adopts the principle of an electrochemical biosensor to detect the blood sugar concentration of a test sample.

At present, there are many sensors used for testing blood sugar, cholesterol, glutamic oxaloacetic transminase (GOT), and glutanic pyruvic transarninase (GPT), etc. Since the strips of these sensors are very small in size and their shapes are very similar, therefore consumers will misuse a sensor strip and thus causing the sensor unable to be used or obtaining a wrong measurement. For example, a cholesterol strip is misused for testing the blood sugar level.

To improve the foregoing shortcomings, the WO Pat. No. 0171328 as shown in FIGS. 1a, 1b and 1c discloses an electrochemical biosensor strip capable of selectively performing a quantitative analysis of blood sugar, cholesterol, and other ingredients in the blood. An end of an electrochemical biosensor strip 50 includes an identifying electrode 60, and the identifying electrode 60 is disposed at different positions according to different applications. In FIG 1a, the identifying electrode 60 is designed on the left side for working with a blood sugar sensor. In FIG. 1b, the identifying electrode 60 is designed on the right side for working with a cholesterol sensor. In FIG 1c, the identifying electrode 60 is designed across both sides for working with a GOT sensor or a GPT sensor.

Although the identifying electrode can be installed at different positions of the strip to work with different sensors to achieve the foregoing objective, yet it is necessary to have different production lines to produce strips of different forms, and the sensor must come with different identification devices. As to manufacturers, such requirement is not cost-effective and cannot lower the cost. As to consumers, the identifying electrode occupies a small area since the size of the strip is very small, and it is difficult to identify the sensor strips. Even though the identifying electrode is changed, the change is still not significant, and it is difficult to precisely identify the strips and easily causing misjudgments.

### SUMMARY OF THE INVENTION

In view of the foregoing shortcomings of the prior art, the inventor of the present invention developed a material having different impedances, and different electrically conductive materials are coated onto the identifying electrode area of the strip, so that when the invention is used for testing, the default impedance of the sensor is compared with the detected impedance of the identifying electrode of the strip. The strip which does not comply with the detection of the sensor will not be able to carry out the test, so that an examiner will not make wrong judgments.

Therefore, it is a primary objective of the invention to provide a biosensor strip having an identification function that comprises a first insulating substrate and a second insulating substrate, and the first substrate is deposited onto the second substrate, and the first substrate includes a working electrode, a reference electrode, and an identifying electrode area, and the working/reference electrodes and the identifying electrode area are exposed from the second substrate, and the identifying electrode area is coated with an electrically conductive material, and the electrically conductive material varies according to different testing applications or different testing instruments. The working electrode and the reference electrode of the first substrate are not in contact with each other, and the other end of the first and second substrates includes a testing area, and the testing area is connected with the working and reference electrodes. When the invention is used and the strip is inserted into the sensor, the impedance produced by the conductive material of the identifying electrode area is used to meet different testing requirements. As a result, manufacturers only need to change the factory default setting of impedance and coat different electrically conductive materials onto the identifying electrode area. Unlike the prior art strip that requires changing the position and form of the identifying electrode to fit different applications or sensors of different specifications, the invention can save manufacturing cost and provide convenient uses.

Another objective of the present invention is to provide a sensor for the sensor strip, and the impedance of the sensor is set to a predetermined range according to the impedance of the sensor strip, when the sensor is manufactured. As a result, the different impedances produced between the electrode and the identifying electrode area is used to determine whether or not the impedance of the sensor falls within the predetermined range; if no, then an error message will be produced to avoid any distortion of the test; if yes, then the biological test sample will be tested. Therefore, the invention can prevent misjudgments, give accurate test results, and provide a foolproof function.

### BRIEF DESCRIPTION OF THE DRAWINGS

To make it easier for our examiner to understand the objective, shape, assembly, structure, characteristics and performance of the present invention, the following embodiments accompanied with the related drawings are described in details.

FIGS 1a, 1b, and 1c are schematic views of WO Pat. No. 0171328;

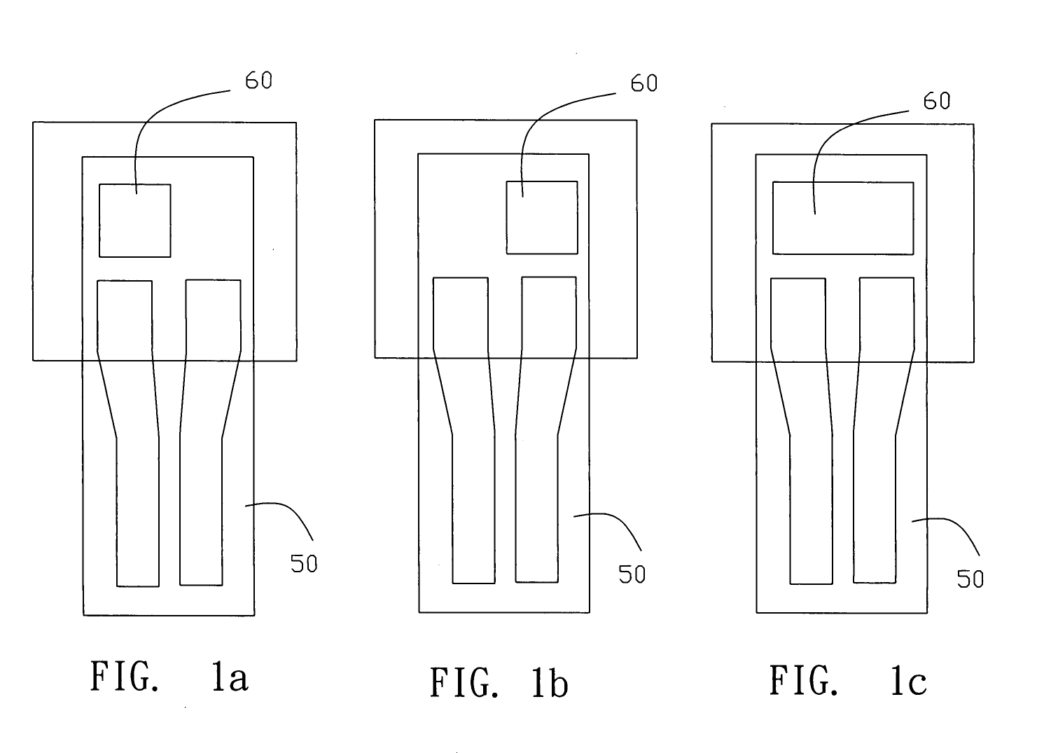

FIG. 2 is a perspective view of a strip of the present invention;

FIG. 2a is an exploded view of a strip of the present invention;

FIG. 2b is an exploded view of working/reference electrodes with respect to an identifying electrode area according to the present invention;

FIG. 3 is a perspective view of a strip according to another preferred embodiment of the present invention;

FIG. 3a is an exploded view of a strip of another preferred embodiment of the present invention;

FIG. 3b is an exploded view of working/reference electrodes with respect to an identifying electrode area according to another preferred embodiment of the present invention; and

FIG. 4 is a schematic view of a sensor of the present invention.

### DETAILED DESCRIPTION OF THE PREFERRED EMBODIMENTS

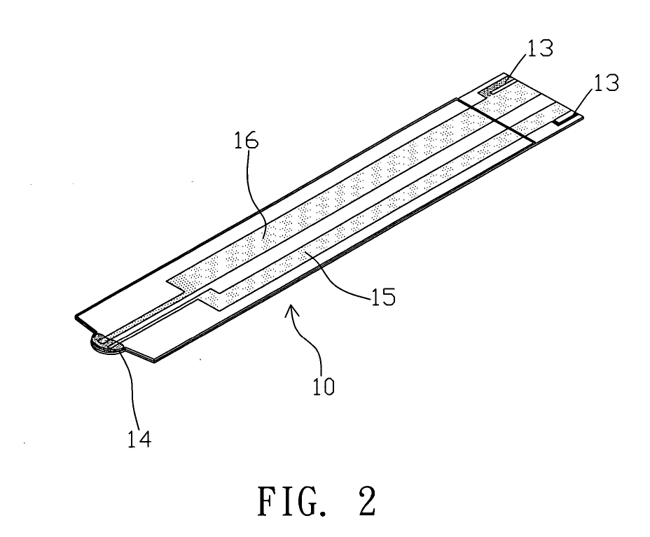

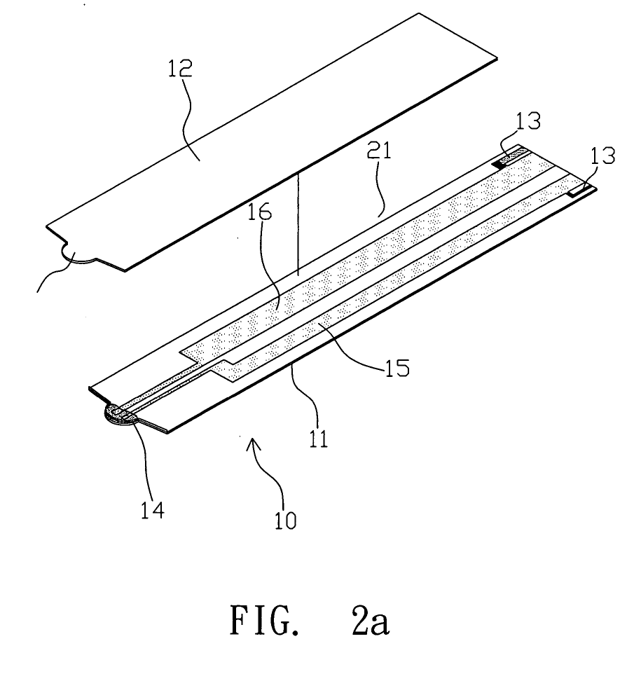

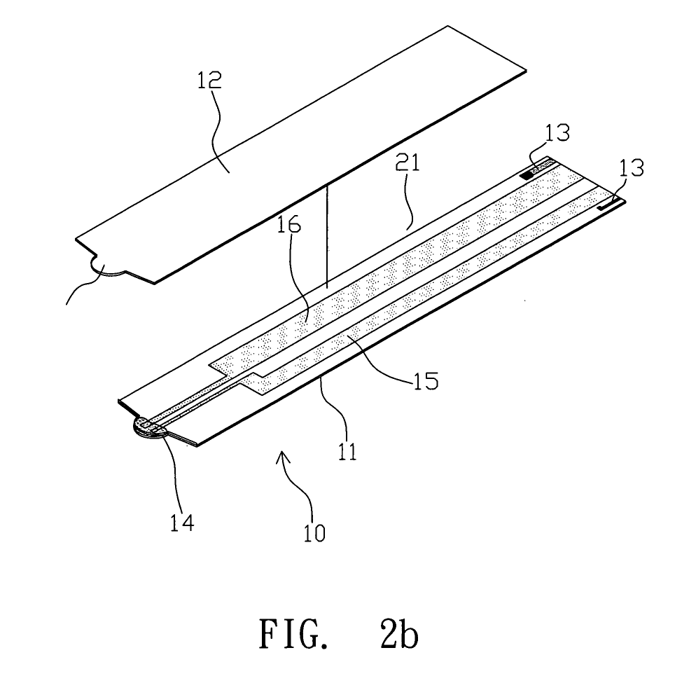

Referring to FIGS. 2 and 2a, the present invention discloses a biosensor strip having an identification function used as a preferred embodiment of an electrochemical biosensor strip, the strip 10 comprises a first substrate 11 and a second substrate 12, and the first substrate 11 is deposited onto the second substrate 12, and the first and second substrates 11, 12 are in a flat sheet shape and electrically insulated. The periphery except both ends of the first and second substrates 11, 12 is coated with an adhesive (it is understood that the persons skilled in the art can use other method to substitute the adhesive to achieve the same effect) to glue the first and second substrates 11, 12 with each other, and the section not covered with the adhesive includes a biological active matter, and the first substrate 11 includes an identifying electrode area 13 at an end having the exposed second substrate 12. The identifying electrode area 13 is coated with an electrically conductive matter. Since different electrically conductive materials have different impedances. For example, a carbon slurry material has an impedance range of 400Ω˜800Ω for a strip, and 1 kΩ˜3kΩ for another strip depending on their compositions, or a rare metal alloy such as platinum or palladium alloys, or a single rare metal such as gold, silver, and platinum, or indium tin oxide.

The first and second substrates 11, 12 include an outwardly protruded testing area 14 on the other end, and a working electrode 15 and a reference electrode 16 disposed between the first and second substrates 11, 12, and maintain a specific distance between the working electrode 15 and the reference electrode 16, and one end of the working electrode 15 and the reference electrode 16 is penetrated deeply into the testing area 14, and the other end and the identifying electrode area 13 are exposed from the second substrate 12 (as shown in FIGS. 2a and 2b).

When the strip is inserted into the sensor, the different impedances produced by the different electrically conductive materials of the identifying electrode area 13, or the impedance produced between one of the electrodes (working electrode 15 or reference electrode 16) and the identifying electrode area 13 cope with different testing requirements or different types of sensors, not only complying with the requirements of different distributing agents, but also allowing manufacturers to coat different electrically conductive materials onto the identifying electrode area 13 according to the factory default setting. Unlike the prior art strip that requires sensors of different specifications to fit different applications or changing the position, form and quantity of the identifying electrode, the invention can save manufacturing cost and provide convenience for its use.

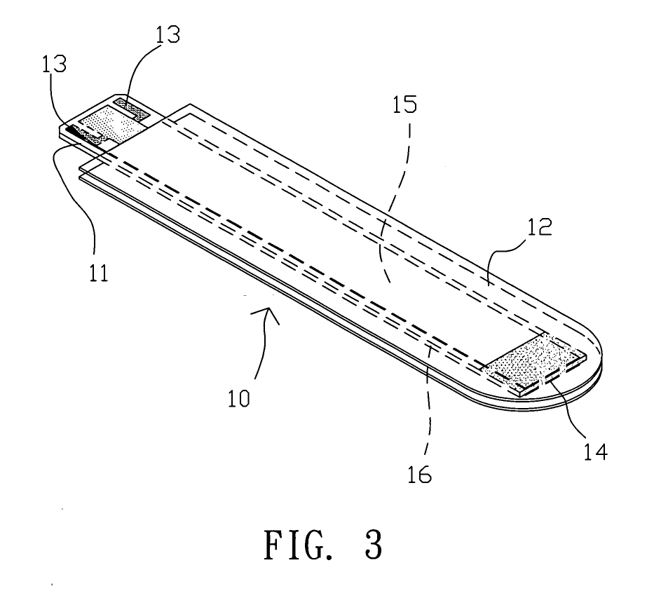

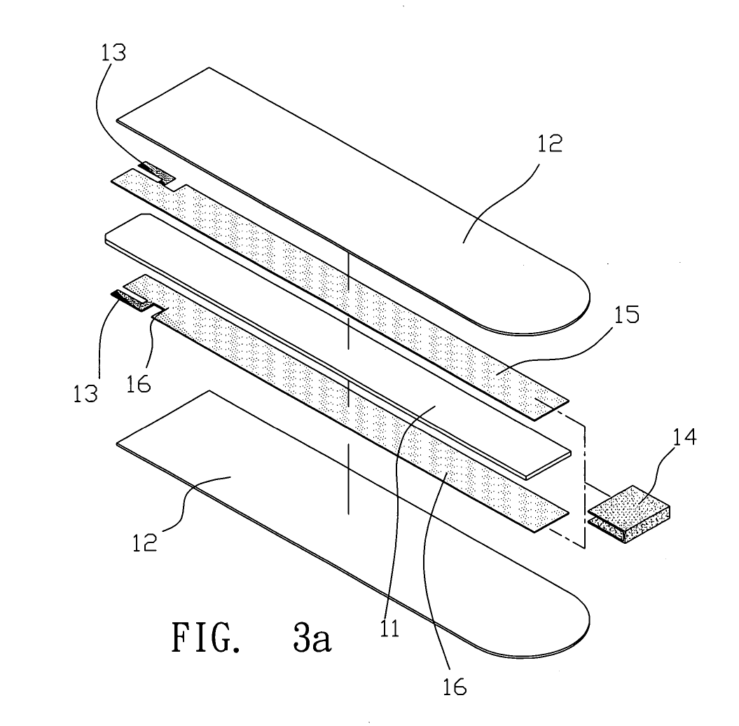

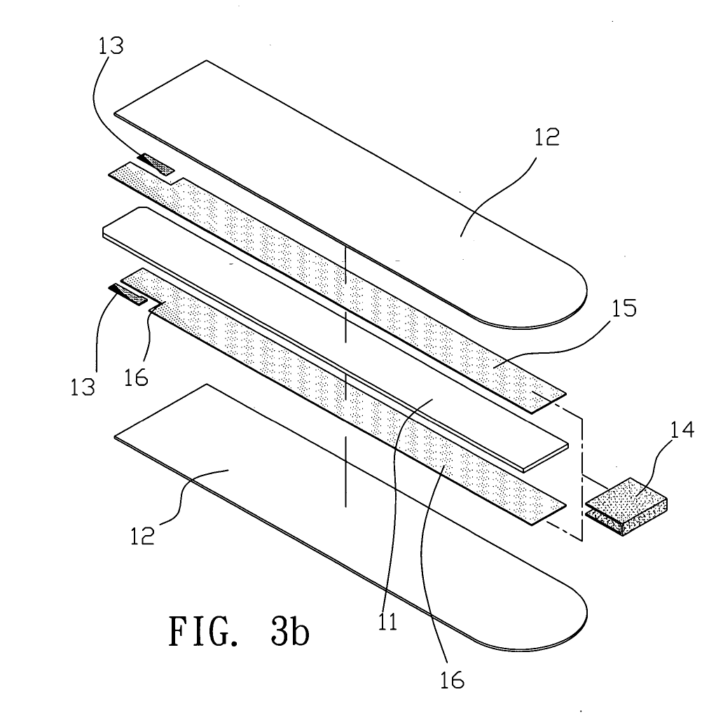

Referring to FIGS. 3 and 3a for the preferred embodiment of an electrochemical strip being used in the present invention, the strip 10 comprises a first substrate 11 and a second substrate 12 disposed on both sides of the first substrate 11, and the first substrate 11 is deposited onto the second substrate 12, and the first and second substrates 11, 12 are in a flat sheet shape and electrically insulated. The periphery except both ends of the first and second substrates 11, 12 is coated with an adhesive (however, the persons skilled in the art can use other methods or equivalents to achieve the same effect) to glue the first and second substrates 11, 12 with each other, and the section without the adhesive covered includes a biologically active matter for testing. The first substrate 11 includes an identifying electrode area 13 disposed at an end having an exposed second substrate 12, and the identifying electrode area 13 is coated with the electrically conductive material. Different electrically conductive materials have different impedances a carbon slurry material has an impedance range of 400Ω˜800Ω for a strip, and 1 kΩ˜3 kΩ for another strip depending on their compositions, or a rare metal alloy such as platinum or palladium alloys, or a single rare metal such as gold, silver, and platinum, or indium tin oxide.

The first and second substrates 11, 12 includes a working electrode 15 and a reference electrode 16 disposed between the first and second substrates 11, 12 and a specific distance and an electrical disconnection between the working electrode 15 and the reference electrode 16 are maintained by the first substrate 11, and one end of the working electrode 15 and the reference electrode 16 is penetrated deeply into the testing area 14, and the other end and the identifying electrode area 13 are exposed from the second substrate 12 (as shown in FIGS. 3a and 3b).

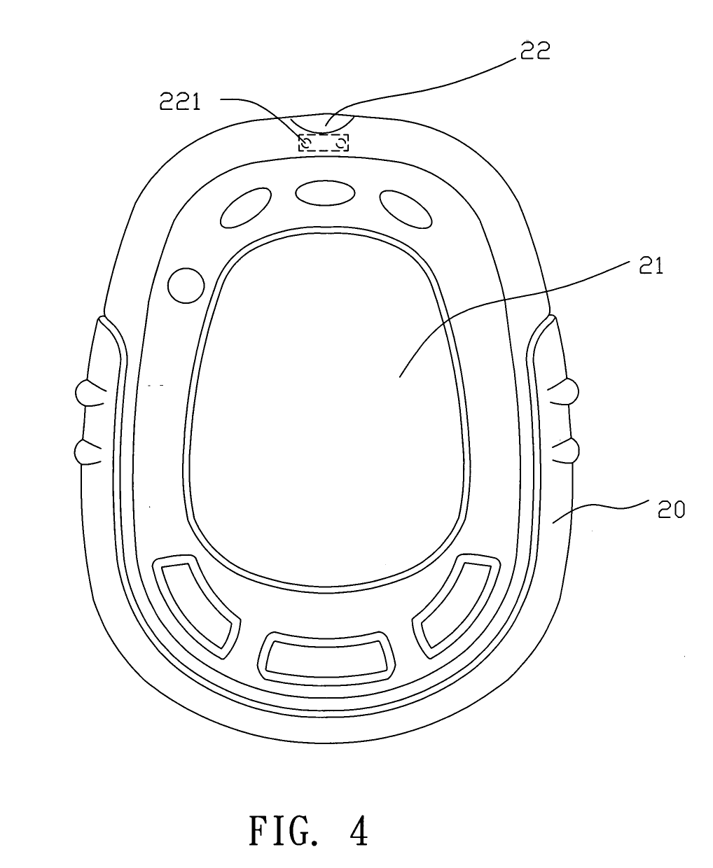

Referring to FIG. 4 for the sensor, the sensor includes a casing 20, and the casing 20 includes a display device 21, and a microprocessor in the casing, and the microprocessor is connected separately with a testing unit, an analog-to-digital converter, a display device 21, a storage unit and a power supply (not shown in the figure). When the invention is used, the testing unit tests a biological test sample, and the analog-to-digital converter coverts data into digital data. In the meantime, the microprocessor uses the input unit to set the standard values into a storage unit for accesses and comparisons, and the processed data is displayed on the display device (these are prior art structures, and thus will not be described here). The casing 20 includes a testing opening 22 at one end, and the testing opening 22 includes a detecting point 221 corresponding to the strip 10. The detecting point 221 is connected to a control system of the sensor (not shown in the figure). The detecting point 221 of the present preferred embodiment can detect the identifying electrode area 13 or the electrode (working electrode 15 or reference electrode 16) on the identifying electrode area 13.

Referring to FIGS. 2 to 4, the strip is inserted into the testing opening 22 before the test is taken, so that the identifying electrode area 13 or the identifying electrode area 13 is in contact with one of the electrodes and the detecting point 221. After the conversion process, the sensor determines whether or not the impedance produced by different electrically conductive materials of the strip identifying electrode area 13 or the impedance produce between the identifying electrode area 13 and one of the electrodes falls within the predetermined range of impedance; if no, then the display device 21will show an error message; if yes, then the test will be continued. The invention not only prevents misjudgments, but also gives accurate test results and provides a foolproof function.

In summation of the above description, the present invention herein enhances the performance and overcomes the shortcoming of the prior art, and further complies with the patent application requirements.

While the invention has been described by way of example and in terms of a preferred embodiment, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A biosensor strip having an identification function, comprising:
an electrically conductive substrate, having a working electrode and a reference electrode;
an identifying electrode area, disposed at an end of said substrate and coated with an electrically conductive material, and said electrically conductive material varies according to different values of impedance; and
a testing area, disposed at another end of said substrate and coupled with said working electrode and said reference electrode.

2. The biosensor strip having an identification function of claim 1, wherein said electrically conductive material is carbon slurry.

3. The biosensor strip having an identification function of claim 1, wherein said electrically conductive material is a rare metal selected from a collection of platinum and palladium alloys, or a single rare metal selected from a collection of gold, silver, or palladium, or indium tin oxide.

4. The biosensor strip having an identification function of claim 1, wherein said substrate includes corresponding first and second substrates, and said first and second substrates is in a flat sheet shape and electrically insulated, and the periphery except both ends of said first and second substrates is coated with an adhesive such that said first and second substrates are glued with each other, and a testing area whose section which is not covered with said adhesive includes a biologically active matter for testing.

5. The biosensor strip having an identification function of claim 1, wherein said substrate includes a first substrate and a second substrate disposed on both sides of said first substrate, and said first and second substrates are in a flat sheet shape and electrically insulated, and the periphery except both ends of said first and second substrates is coated with an adhesive, such that said first and second substrates are glued with each other, and a testing area whose section not covered with said adhesive includes a biologically active matter for testing.

6. A sensor for biosensor strip having an identification function, comprising a testing opening, and said testing opening includes a testing point for a detection, and said sensor sets a predetermined range of impedance, so that if said strip is inserted into said sensor, said sensor can determine whether or not the impedance produced by different electrically conductive materials on said strip identifying electrode falls within said predetermined range of impedance; if no, then an error message will be produced; and if yes, then a test will be performed, so as to prevent misjudgments, obtain accurate test results, and provide a foolproof function.

7. The sensor of claim 6, wherein said testing point includes a detecting point disposed in said identifying electrode area.

8. The sensor of claim 6, wherein said testing point includes a detecting point disposed at a relative position of said identifying electrode area and said working electrode or said reference electrode.
